SEQUENCE LISTING (10 sequences)

SEQ ID NO: 1 - RNA, 21 nt, Hepatitis C virus
agacagucgacuucagccugg

SEQ ID NO: 2 - RNA, 21 nt, Hepatitis C virus
aggcugaagucgacugucugg

SEQ ID NO: 3 - RNA, 22 nt, Artificial sequence
  Description of the artificial sequence vector
cuacguccaggagcgcaccauc

SEQ ID NO: 4 - RNA, 24 nt, Artificial sequence
  Description of the artificial sequence vector
gauggugcgcuccuggacguagcc

SEQ ID NO: 5 - DNA, 30 bp, Hepatitis C virus
cattgtcgtagcaacggacgctctaatgac

SEQ ID NO: 6 - DNA, 27 bp, Hepatitis C virus
ccttgatgtatccgtcataccaactag

SEQ ID NO: 7 - DNA, 20 bp, Hepatitis C virus
tgagtcgaaatcgccggtaa

SEQ ID NO: 8 - DNA, 27 bp, Homo sapiens
aaccgtcacctgggaccgagacatgta

SEQ ID NO: 9 - DNA, 25 bp, Homo sapiens
ccgatgtatatgcttgcagagttaa

SEQ ID NO: 10 - DNA, 23 bp, Homo sapiens
cagatgattcagagctccataga

(19) United States
(12) Patent Application Publication
(10) Pub. No.: US 2004/0248835 A1
(43) Pub. Date: Dec. 9, 2004

(54) Use of a double-stranded ribonucleic acid for treating an infection with a positivestrand rna-virus

(76) Inventors: Anja Krebs, Nurnberg (DE); Matthias John, Hallstadt (DE); Detlef Schuppan, Bubenreuth (DE); Stefan Limmer, Kulmbach (DE); Roland Kreutzer, Weidenberg (DE)

Correspondence Address: RANKIN, HILL, PORTER & CLARK LLP, 4080 ERIE STREET, WILLOUGHBY, OH, 44094-7836, US

(21) Appl. No.: 10/493,768

(22) PCT Filed: Oct. 25, 2002
(86) PCT No.: PCT/EP02/11973

(30) Foreign Application Priority Data
Oct. 26, 2001 (DE) 10155280.7

(51) Int. Cl. A61K 48/00

(52) U.S. Cl. 514/44; 435/456

(57) ABSTRACT

The invention concerns the use of a double-stranded ribonucleic acid (dsRNA) to treat a (+) strand RNA virus infection, wherein one strand S1 of the dsRNA exhibits a region that is at least segmentally complementary to a segment of the translatable region of the virus genome.

The invention concerns the use of a double-stranded ribonucleic acid to treat a (+) strand RNA virus infection, and the use of such a ribonucleic acid to produce a medicament, a medicament and a method to inhibit replication of a (+) strand RNA virus.

A method for inhibiting expression of a target gene in a cell is known from DE 101 00 586 C1, in which an oligoribonucleotide having a double-stranded structure is introduced into the cell. One strand of the double-stranded structure is here complementary to the target gene.

As carriers of genetic information, (+) strand RNA viruses exhibits RNA at which protein synthesis may take place directly in the cell interior. This makes transcription unnecessary. Except for an untranslated 3′- and 5¹-region, the entire length of the virus genome is translated into a polyprotein. Individual, functionally active structural and nonstructural proteins emerge from the polyproteins as a result of cleavages. Non-structural protein sequences follow the structural protein sequences in the viral genome. The non-structural NS3 protein is a multifunctional enzyme with a serine protease domain and exhibits NTPase- and Helicase activity. Previous approaches to treatment of (+) strand RNA virus infections have met with little success, and in most infected patients have led to no lasting improvement of the state of disease.

The task of the present invention is to remove these short-comings in accordance with the state-of-the-art. In particular, an effective use to treat a (+) strand RNA virus infection is to be made available. Furthermore, a medicament to treat a (+) strand RNA virus infection as well as a use to produce such a medicament are to be made available. Furthermore, a method to inhibit the replication of a (+) strand RNA virus is to be made available.

This task is solved by the features in claims 1, 2, 16, 30, and 41. Advantageous embodiments result from the features in claims 3 to 15, 17 to 29, 31 to 40, and 42 to 53.

According to the invention a use of a double-stranded ribonucleic acid (dsRNA) to treat a (+) strand RNA virus infection is intended, whereby one strand S1 of the dsRNA exhibits a region that is at least segmentally complementary to a segment of the translatable region of the virus genome. Furthermore, the invention concerns the use of such dsRNA to produce a medicament to treat a (+) strand RNA virus infection.

It does not matter which is the section of the translatable region of the virus genome. Surprisingly, although the virus genome codes for numerous proteins, it is sufficient for an inhibition of the replication of the (+) strand RNA virus when a dsRNA is used with a strand S1 that is complementary to an arbitrary segment of the translatable region of the virus genome. Such dsRNA can permanently destroy the integrity of the viral RNA genome by means of RNA interference. For this reason, it is ideally suited to treat such a viral infection. Treatment results in lasting improvement of the state of disease.

The (+) strand RNA virus can be a hepatitis C virus (HCV). An effective treatment in this area would be particularly important because to date it has not been possible to produce an effective vaccine against the hepatitis C virus. In humans, HVC-infection can lead to serious diseases, particularly via chronic hepatitis to cirrhosis of the liver and liver cancer.

In an infected cell, the dsRNA causes the (+) strand RNA of the (+) strand RNA virus to be cut enzymatically in the region of the aforementioned segment. The regions in reading direction of the viral RNA before the cleavage site can still be translated, and can at least in part lead to functional proteins. Expression of these proteins is not necessarily inhibited. In an advantageous embodiment of this method, the dsRNA is able to inhibit the expression of a polyprotein coded from the virus genome. Partial inhibition can also ensue, i.e., so that only a portion of the complete polyprotein is expressed, or so that the total quantity of expressed polyproteins is reduced.

DsRNA is preferably able to inhibit the expression of a functional protease or helicase coded from the virus genome, particularly the HCV-NS3 helicase. For that, the segment to which the strand S1 of the dsRNA is complementary can be arranged in reading direction of the viral RNA, in front of or in the virus genome region that codes for the helicase. Surprisingly, inhibition of expression of viral helicase is particularly advantageous. The inventors have discovered that the presence of the viral helicase reduces the replication-inhibiting action of dsRNA. Because of inhibition of the expression of helicase, the action of dsRNA is stronger than is the case in inhibition of the expression of other viral proteins

The complementary region of the dsRNA may exhibit-in order of ascending preference-fewer than 25, 19 to 24, 20 to 24, 21 to 23, and particularly 22 or 23 nucleotides. DsRNA having this structure is particularly efficient in treating virus infection, and especially in inhibiting virus replication. The strand S1 of the dsRNA can exhibit-in order of ascending preference-fewer than 30, fewer than 25, 21 to 24, and particularly 23 nucleotides. The number of these nucleotides is also the maximum number of possible base pairs in the dsRNA. Such dsRNA is particularly stable intracellularly.

DsRNA preferably exhibits a single stranded overhang consisting of 1 to 4, particularly 2 or 3 nucleotides at least at one end of the dsRNA. Single stranded overhangs reduce the stability of the dsRNA in blood, serum, and cells, while at the same time increasing the replication-inhibiting action of the dsRNA. It is particularly advantageous when the dsRNA exhibits the overhang exclusively at one end, in particular at its end that exhibits the 3′-end of the strand S1. At a dsRNA that exhibits two ends the other end is then blunt, i.e., lacks overhangs. Surprisingly, it has been shown that to increase the replication-inhibiting action of the dsRNA, one overhang at one end of the dsRNA is sufficient, and does not decrease stability to such an extent as occurs with two over-hangs. DsRNA with only one overhang has shown itself to be sufficiently stable and particularly effective in various cell culture mediums, as well as in blood, serum, and cells. Inhibition of the replication of viruses is particularly effective when the overhang is located at the 3′-end of the strand S1.

Preferably, the dsRNA exhibits a strand S2 in addition to the strand S1, i.e., it is comprised of two individual strands. DsRNA is particularly effective when the strand S1 (antisense strand) is 23 nucleotides long, the strand S2 is 21 nucleotides long, and the 31-end of the strand S1 exhibits a single stranded overhang made up of two nucleotides. Here the dsRNA end located at the 5′-end of the strand S1 is blunt.

The dsRNA may be present in a preparation suitable to be administered orally, by inhalation, infusion and injection, in particular intravenous or intraperitoneal infusion or injection. This preparation can consist, in particular exclusively, of the dsRNA and a physiologically tolerated solvent, preferably a physiological saline solution or a physiologically tolerated buffer. The physiologically tolerated buffer may be a phosphate buffered saline solution. Surprisingly, it has been shown that dsRNA that has simply been dissolved and administered in such a solvent or such a buffer is taken up by cells and inhibits expression of a target gene or replication of a virus, without the dsRNA having had to be packaged in a special vehicle.

Preferably, the dsRNA is present in a physiologically tolerated solution, particularly in a physiologically tolerated buffer or physiological saline solution, or surrounded by a micellar structure, preferably a liposome, a virus capsid, a capsoid, or a polymeric nano- or microcapsule, or bound to a polymeric nano- or microcapsule. The physiologically tolerated buffer can be a phosphate buffered saline solution. A micellar structure, a virus capsid, capsoid, or polymeric nano- or microcapsule can facilitate uptake of the dsRNA in infected cells. The polymeric nano- or microcapsule consists of at least one biologically degradable polymer such as poly-butylcyanoacrylate. The polymeric nano- or microcapsule can transport and release in the body dsRNA that is contained in or bound to it. The dsRNA may be administered or taken orally, by means of inhalation, infusion, or injection, in particular by intravenous or intraperitoneal infusion or injection.

Preferably, the dsRNA is used in a dosage of—in order of ascending preference—maximal 5 mg, 2.5 mg, 200 μg, 100 μg, 50 μg, and optimally maximal 25 μg per kg body weight per day. It has been shown that the dsRNA exhibits outstanding effectiveness even at this dosage in the treatment of a (+) strand RNA virus infection.

Furthermore, the invention concerns a medicament to treat a (+) strand RNA virus infection, whereby the medicament contains a double-stranded ribonucleic acid (dsRNA), in which one strand S1 exhibits a region that is at least segmentally complementary to a segment of the translatable region of the virus genome. Preferably, the medicament is available in at least one dosage unit that contains the dsRNA in a quantity that makes possible—in order of ascending preference—a maximum dosage of 5 mg, 2.5 mg, 200 μg, 100 μg, 50 μg, and optimally 25 μg per kilogram body weight per day. The dosage unit can be compounded for single daily dose administration or ingestion. In this case, the entire daily dose is contained in a single dosage unit. If the dosage unit is compounded to be administered or ingested several times per day, the quantity of dsRNA contained in each dose is correspondingly smaller in order to achieve the total daily dosage. The dosage unit can also be compounded for a single administration or ingestion over several days, e.g., so that the dsRNA is released over several days. The dosage unit then contains a corresponding multiple of the daily dose.

Furthermore, according to the invention a method to inhibit replication of a (+) strand RNA virus in a cell is intended, whereby at least one double-stranded ribonucleic acid (dsRNA) is introduced into the cell, and whereby one strand S1 of the dsRNA exhibits a region that is at least segmentally complementary to a segment of the translatable region of the virus genome. The invention furthermore concerns a dsRNA, in which a strand S1 of the dsRNA exhibits a region that is at least segmentally complementary to a segment of the translatable region of the (+) strand RNA virus genome.

With regard to further advantageous embodiments of the medicament, method, and dsRNA according to the invention, see the previous remarks. Subsequently, the invention will be explained exemplary using the figure.

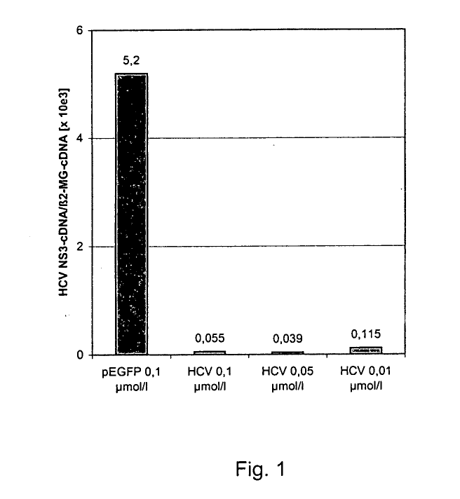

FIG. 1 shows a graphic representation of the reduction of HCV-RNA in the HCV replicon model by means of transfection of NS3-specific dsRNA.

HCV has a genome with approximately 9600 nucleotides. It codes for the structural proteins C, E1, and E2, and for the non-structural proteins NS2, NS3, NS4a, NS4b, NS5a, and NS5b. Because molecular-biological analysis with HCV in cell culture are very difficult, the action of dsRNA on viral gene sequences is studied by means of a non-pathogenic substitute system. For this, a neomycin-resistance-mediating neomycin cassette replaces the part of the viral genome that codes for structural proteins C, E1, and E2. The modified viral genome is registered under Gene Accession No. AJ242654 with the National Center for Biotechnology Information (NCBI), National Library of Medicine, Building 38A, Bethesda, Md. 20894, USA. It has been transfected in HuH-7 liver cells (JCRB0403, Japanese Collection of Research Bioresources Cell Bank, National Institute of Health Sciences, Kamiyoga, 1-18-1, Setagaya-ku, Tokyo 158, Japan). It replicates in these cells in the presence of the neomycin analog G418, without allowing infectious particles to be created. The system that makes possible stable replication of the modified HCV genome (Lohmann et al. Science 285, [1999], page 110) is also designated as the “replicon model” for hepatitis C viruses.

The RNAs used exhibit the following sequences, designated as SEQ ID NO:1 to SEQ ID NO:4 in the sequence listing:

dsRNA1, which corresponds to a sequence from the region that codes for NS3

| | |
|---|---|
| S2: | |
| 5′- AGA CAG UCG ACU UCA GCC UGG-3′ | (SEQ ID NO: 1) |
| S1: | |
| 3′-GG UCU GUC AGC UGA AGU CGG A-5′ | (SEQ ID NO: 2) |

dsRNA2, which, as the negative control with no relation to the NS3 sequence, corresponds to the sequence of the nucleotides 886-909 of the pEGFP-C1 vector, Accession No. U55763, NCBI:

| | |
|---|---|
| S2: | |
| 5′- CUA CGU CCA GGA GCG CAC CA | (SEQ ID NO: 3) |
| UC-3′ | |
| S1: | |
| 3′-CC GAU GCA GGU CCU CGC GUG GU | (SEQ ID NO: 4) |
| AG-5′ | |

In each case, S2 represents the sense strand and S1 the antisense strand, i.e., the sequence of the strand S2 is identical to the corresponding sequence from the HCV.

The HuH 7 cells are cultivated in the presence of 1 mg/ml of the antibiotic G418 in Dulbecco's modified Eagle's Medium with 20% fetal calf serum. For transfection, 80,000 cells per well (3.5 cm diameter) of a six-well plate are seeded in 2 ml of medium. “Fugene 6” (Catalog No. 1814443), Roche Diagnostics GmbH, Sandhofer Str. 116, 68305 Mannheim, Germany, was used to aid transfection in accordance with the accompanying instructions. For this, 100 μl serum-free medium (SFM) was mixed in a reagent vessel with 5 μl Fugene 6 reagent, and incubated for 5 minutes at room temperature. 3 μg dsRNA2 (corresponds to approximately 0.1 μmol/l final dsRNA2 concentration), 3 μg dsRNA1 (corresponds to approximately 0.1 μmol/l final dsRNA1 concentration), 1.5 ↑g dsRNA1 plus 1.5 μg dsRNA2 (corresponds to approximately 0.05 μmol/l final dsRNA1 concentration), or 300 ng dsRNA1 plus 2.7 μg dsRNA2 (corresponds to approximately 0.01 μmol/l final dsRNA1 concentration) were prepared in other reagent vessels each. In each case, the stock concentration of dsRNA1 and dsRNA2 was equal to 20 μM (corresponding to approximately 300 ng/μl). The mixture made up of Fugene 6 and SFM was added drop by drop to the nucleic acids, mixed carefully with a tip of a pipette, and incubated for 15 minutes at room temperature. For transfection, the reaction preparation was added drop by drop to the cells. Each transfection was done at least twice, and verified in at least 2 independent experiments.

The action of dsRNA on the replication of the modified HCV genome was determined by means of quantitative PCR. Approximately 36 hours after transfection, the cells were disintegrated, and the RNA they contained was isolated with a PeqGold RNAPure kit (PEQLAB Biotechnology GmbH, Carl-Thiersch-Str. 2b, 91052 Erlangen, Germany, Order No. 30-1010) in accordance with manufacturer instructions.

Subsequently, the same quantities of RNA (100-1000 ng) were used for reverse transcription, using Superscript II (Invitrogen GmbH, Karlsruhe Technology Park, Emmy-Noether-Str. 10, 76131 Karlsruhe, Germany, catalogue number 18064-014). 100 pmol oligo-dT primer or 50 pmol random primer were used as primers. 10 μl RNA (100-1000 ng), 0.5 Al oligo-dT primer (100 pmol), and 1 μl random primer (50 pmol) were incubated for 10 minutes at 70° C., and then stored on ice for a short time. Then 7 μl reverse transcriptase mix (4 μl 5× buffer; 2 μl 0.1 mol/l DTT; 1 μl each per 10 mmol/l dNTP), 1 μl Superscript II, and 1 μl of the ribonuclease inhibitor RNAsin® [Promega GmbH, Schildkrötstr. 15, 68199 Mannheim, Germany] was added. The mixture was kept for 10 minutes at 25° C., then for 1 hour at 42° C., and finally for 15 minutes at 70° C.

Specific cDNA quantities were quantified from the same volumes of cDNA formed in a “Light-Cycler” (Roche Diagnostics GmbH) according to the “TaqMan” method (PerkinElmer, Ferdinand-Porsche-Ring 17, 63110 Rodgau-Jügesheim, Germany) in accordance with manufacturer instructions, using the LightCycler Fast Start DNA Master Hybridization Probes kit (Roche Diagnostics GmbH). Detection was done by means of a probe that was marked at the 5′-end with the fluorophore 6′-FAM (carboxyfluorescein) and at the 3′-end with the quencher molecule TAMRA (carboxy-tetra-methyl-rhodamine). The fluorophore is stimulated by light and transfers the stimulus energy to the 3′-sided quencher molecule that is in its immediate vicinity. During each of the extension phases of PCR reaction, the 5′-3′ exonuclease activity of the Taq DNA polymerase leads to hydrolysis of the probe, and with it to spatial separation of the fluorophore from the quencher molecule. The fluorescence of 6′-FAM is progressively less quenched. Because of this, it increases and is quantitatively determined

The following were used for quantification of HCV NS3-cDNA:

| | |
|---|---|
| NS3 probe: | |
| 5′-CAT TGT CGT AGC AAC GGA CGC TCT | (SEQ ID NO 5) |
| AAT GAC-3′ | |
| NS3 primer: | |
| 5′-CCT TGA TGT ATC CGT CAT ACC AAC | (SEQ ID NO 6) |
| TAG-3′ | |
| NS3 reverse primer: | |
| 5′-TGA GTC GAA ATC GCC GGT AA-3′ | (SEQ ID NO 7) |

Furthermore, β2-microglobulin cDNA was quantified as the standard. β2-microglobulin (β2-MG) is a protein that is expressed constitutively in a steady quantity. The following were used for quantification:

| | |
|---|---|
| β2-microglobulin probe: | |
| 5′-AAC CGT CAC CTG GGA CCG AGA CAT | (SEQ ID NO 8) |
| GTA-3′ | |
| β2-microglobulin primer: | |
| 5′-CCG ATG TAT ATG CTT GCA GAG TTA | (SEQ ID NO 9) |
| A-3′ | |
| β2-microglobulin reverse primer: | |
| 5′-CAG ATG ATT CAG AGC TCC ATA | (SEQ ID NO 10) |
| GA-3′ | |

The NS3 probe and the β2-microglobulin probe each exhibited FAM marking at the 5′-end, and TAMRA marking at the 3′-end.

The quantity of HCV NS3 cDNA was determined in form of the ratio to the quantity of β2-MG cDNA and is represented graphically in FIG. 1. “pEGFP” represents the value determined by transfection exclusively with dsRNA2 (control), and “HCV 0.1 μmol/l,” “HCV 0.05 μmol/l,” and “HCV 0.01 μmol/1” represent the values determined by transfection with NS3-specific dsRNA1 with 0.1 μmol/l, 0.05 μmol/l, and 0.01 μmol/l, respectively.

At a final concentration of 0.1 μmol/l, 0.05 μmol/l, and 0.01 μmol/l in medium, transfection with dsRNA1 lead to an approximately 60-fold greater inhibition in comparison to transfection with dsRNA2, the non-specific control.

1. Use of a double-stranded ribonucleic acid (dsRNA) to treat a (+) strand RNA virus infection, wherein one strand S1 of the dsRNA exhibits a region that is at least segmentally complementary to a segment of the translatable region of the virus genome, and wherein the dsRNA is able to inhibit the expression of a functional protease or helicase coded from the virus genome.

2. Use of a double-stranded ribonucleic acid (dsRNA) to produce a medicament to treat a (+) strand RNA virus infection, wherein one strand S1 of the dsRNA exhibits a region that is at least segmentally complementary to a segment of the translatable region of the virus genome, and wherein the dsRNA is able to inhibit the expression of a functional protease or helicase coded from the virus genome.

3. Use in accordance with claim 1, wherein the(+) strand RNA virus is a hepatitis C virus (HCV).

4. Use in accordance with claim 1, wherein the dsRNA is able to inhibit the expression of a polyprotein coded from the virus genome.

5. Use in accordance with claim 1, wherein the helicase is the HCV-NS3 helicase.

6. Use in accordance with claim 1, wherein the segment in reading direction of the viral RNA is arranged in front of or in the region of the virus genome that codes for helicase, particularly HCV-NS3 helicase.

7. Use in accordance with claim 1, wherein the complementary region exhibits—in order of ascending preference—fewer than 25, 19 to 24, 20 to 24, 21 to 23, and particularly 22 or 23 nucleotides.

8. Use in accordance with claim 1, wherein the strand S1 exhibits—in order of ascending preference—fewer than 30, fewer than 25, 21 to 24, and particularly 23 nucleotides.

9. Use in accordance with claim 1, wherein the dsRNA exhibits a single stranded overhang consisting of 1 to 4, particularly 2 or 3 nucleotides at least at one end of the dsRNA.

10. Use in accordance with claim 9, wherein the dsRNA exhibits the overhang exclusively at one end, in particular at its end that exhibits the 3′-end of the strand S1.

11. Use in accordance with claim 1, wherein the dsRNA exhibits a strand S2 in addition to the strand S1, and the strand S1 is 23 nucleotides long, the strand S2 is 21 nucleotides long, and the 3′-end of the strand S1 exhibits a single stranded overhang made up of two nucleotides, while the dsRNA end located at the 5′-end of the strand S1 is blunt.

12. Use in accordance with claim 1, wherein the dsRNA is present in a preparation suitable to be administered orally, by means of inhalation, infusion, or injection, in particular intravenous or intraperitoneal infusion or injection.

13. Use in accordance with claim 1 wherein the preparation consists, particularly exclusively, of the dsRNA and a physiologically tolerated solvent, preferably a physiological saline solution or a physiologically tolerated buffer, in particular a phosphate buffered saline solution.

14. Use in accordance with claim 1, wherein the dsRNA is present in a physiologically tolerated solution, particularly in a physiologically tolerated buffer or physiological saline solution, or surrounded by a micellar structure, preferably a liposome, a virus capsid, a capsoid, or a polymeric nano- or microcapsule, or bound to a polymeric nano- or microcapsule.

15. Use in accordance with claim 1, wherein the dsRNA is used in a dosage of—in order of ascending preference—maximal 5 mg, 2.5 mg, 200 μg, 100 μg, 50 μg, and optimally maximal 25 μg per kg body weight per day.

16. Medicament to treat a (+) strand RNA virus infection, wherein the medicament contains a double-stranded ribonucleic acid (dsRNA) in which one strand S1 of the dsRNA exhibits a region that is at least segmentally complementary to a segment of the translatable region of the virus genome, wherein the dsRNA is able to inhibit the expression of a functional protease or helicase coded from the virus genome.

17. Medicament in accordance with claim 16, wherein the(+) strand RNA virus is a hepatitis C virus (HCV).

18. Medicament in accordance with claim 16, wherein the dsRNA is able to inhibit the expression of a polyprotein coded from the virus genome.

19. Medicament in accordance with claim 16, wherein the helicase is the HCV-NS3 helicase.

20. Medicament in accordance with claim 16, wherein the segment in reading direction of the viral RNA is arranged in front of or in the region of the virus genome that codes for helicase, particularly HCV-NS3 helicase.

21. Medicament in accordance with claim 16, wherein the complementary region exhibits—in order of ascending preference—fewer than 25, 19 to 24, 20 to 24, 21 to 23, and particularly 22 or 23 nucleotides.

22. Medicament in accordance with claim 16, wherein the strand S1 exhibits—in order of ascending preference—fewer than 30, fewer than 25, 21 to 24, and particularly 23 nucleotides.

23. Medicament in accordance with claim 16, wherein the dsRNA exhibits a single stranded overhang consisting of 1 to 4, in particular 2 or 3 nucleotides at least at one end of the dsRNA.

24. Medicament in accordance with claim 23, wherein the dsRNA exhibits the overhang exclusively at one end, in particular at its end that exhibits the 3′-end of the strand S1.

25. Medicament in accordance with claim 16, wherein the dsRNA exhibits a strand S2 in addition to the strand S1, and the strand S1 is 23 nucleotides long, the strand S2 is 21 nucleotides long, and the 3′-end of the strand S1 exhibits a single stranded overhang made up of two nucleotides, while the dsRNA end located at the 5′-end of the strand S1 is blunt.

26. Medicament in accordance with claim 16, wherein the medicament exhibits a preparation suitable to be administered orally, by means of inhalation, infusion, or injection, in particular by intravenous or intraperitoneal infusion or injection.

27. Medicament in accordance with claim 26, wherein the preparation consists, particularly exclusively, of the dsRNA and a physiologically tolerated solvent, preferably a physiological saline solution or a physiologically tolerated buffer, particularly a phosphate buffered saline solution.

28. Medicament in accordance with claim 16, wherein the dsRNA is present in the medicament in a solution, particularly in a physiologically tolerated buffer or physiological saline solution, or surrounded by a micellar structure, preferably a liposome, a virus capsid, a capsoid, or a polymeric nano- or microcapsule, or bound to a polymeric nano- or microcapsule.

29. Medicament in accordance with claim 16, wherein the medicament is available in at least one dosage unit that contains the dsRNA a quantity that makes possible—in order of ascending preference—a maximum dosage of 5 mg, 2.5 mg, 200 μg, 100 μg, 50 μg, and optimally 25 μg per kg body weight per day.

30. Method to inhibit replication of a (+) strand RNA virus in a cell, wherein at least one double-stranded ribonucleic acid (dsRNA) is introduced into the cell, and whereby one strand S1 of the dsRNA exhibits a region that is at least segmentally complementary to a segment of the translatable region of the virus genome, wherein the expression of a functional protease or helicase coded from the virus genome is inhibited.

31. Method in accordance with claim 30, wherein the(+) strand RNA virus is a hepatitis C virus.

32. Method in accordance with claim 30, wherein the expression of a polyprotein coded from the virus genome is inhibited.

33. Method in accordance with claim 30, wherein the helicase is the HCV-NS3 helicase.

34. Method in accordance with claim 33, wherein the segment in reading direction of the viral RNA is arranged in front of or in the region of the virus genome that codes for helicase, particularly HCV-NS3 helicase.

35. Method in accordance with claim 30, wherein the complementary region exhibits—in order of ascending preference—fewer than 25, 19 to 24, 20 to 24, 21 to 23, and particularly 22 or 23 nucleotides.

36. Method in accordance with claim 30, wherein the strand S1 exhibits—in order of ascending preference—fewer than 30, fewer than 25, 21 to 24, and in particular 23 nucleotides.

37. Method in accordance with claim 30, wherein the dsRNA exhibits a single stranded overhang consisting of 1 to 4, particularly 2 or 3 nucleotides at least at one end of the dsRNA.

38. Method in accordance with claim 37, wherein the dsRNA exhibits the overhang exclusively at one end, in particular at its end that exhibits the 3′-end of the strand S1.

39. Method in accordance with claim 30, wherein the dsRNA exhibits a strand S2 in addition to the strand S1, and the strand S1 is 23 nucleotides long, the strand S2 is 21 nucleotides long, and the 3′-end of the strand S1 exhibits a single stranded overhang made up of two nucleotides, while the dsRNA end located at the 5′-end of the strand S1 is blunt.

40. Method in accordance with claim 30, wherein the dsRNA is present in a solution, particularly in a physiologically tolerated buffer or physiological saline solution, or surrounded by a micellar structure, preferably a liposome, a virus capsid, a capsoid, or a polymeric nano- or microcapsule, or bound to a polymeric nano- or microcapsule.

41. Double-stranded ribonucleic acid (dsRNA) in which a strand S1 of the dsRNA exhibits a region that is at least segmentally complementary to a segment of the translatable region of the genome of a (+) strand RNA virus, wherein the dsRNA is able to inhibit the expression of a functional protease or helicase coded from the virus genome.

42. DsRNA in accordance with claim 41, wherein the(+) strand RNA virus is a hepatitis C virus.

43. DsRNA in accordance with claim 41, wherein the dsRNA is able to inhibit the expression of a polyprotein coded from the virus genome.

44. DsRNA in accordance with claim 41, wherein the helicase is the HCV-NS3 helicase.

45. DsRNA in accordance with claim 41, wherein the segment in reading direction of the viral RNA is arranged in front of or in the region of the virus genome that codes for helicase, particularly HCV-NS3 helicase.

46. DsRNA in accordance with claim 41, wherein the complementary region exhibits—in order of ascending preference—fewer than 25, 19 to 24, 20 to 24, 21 to 23, and particularly 22 or 23 nucleotides.

47. DsRNA in accordance with claim 41, wherein the strand S1 exhibits—in order of ascending preference—fewer than 30, fewer than 25, 21 to 24, and particularly 23 nucleotides.

48. DsRNA in accordance with claim 41, wherein the dsRNA exhibits a single stranded overhang consisting of 1 to 4, particularly 2 or 3 nucleotides at least at one end of the dsRNA.

49. DsRNA in accordance with claim 48, wherein the dsRNA exhibits the overhang exclusively at one end, in particular at its end that exhibits the 3′-end of the strand S1.

50. DsRNA in accordance with claim 41, wherein the dsRNA exhibits a strand S2 in addition to the strand S1, and the strand S1 is 23 nucleotides long, the strand S2 is 21 nucleotides long, and the 3′-end of the strand S1 exhibits a single stranded overhang made up of two nucleotides, while the dsRNA end located at the 5′-end of the strand S1 is blunt.

51. DsRNA in accordance with claim 41, wherein the dsRNA is present in a preparation suitable to be administered orally, by means of inhalation, infusion, or injection, particularly intravenous or intraperitoneal infusion or injection.

52. DsRNA in accordance to claim 51, wherein the preparation consists, particularly exclusively, of the dsRNA and a physiologically tolerated solvent, preferably a physiological saline solution or a physiologically tolerated buffer, particularly a phosphate buffered saline solution.

53. DsRNA in accordance with claim 41, wherein the dsRNA is present in a solution, particularly in a physiologically tolerated buffer or physiological saline solution, or surrounded by a micellar structure, preferably a liposome, a virus capsid, a capsoid, or a polymeric nano- or microcapsule, or bound to a polymeric nano- or microcapsule.
